(19) United States
(12) Patent Application Publication
(10) Pub. No.: US 2005/0177131 A1
(43) Pub. Date: Aug. 11, 2005

(54) Catheter articulation segment with alternating cuts

(76) Inventors: David J. Lentz, La Jolla, CA (US); Richard J. Koerner, San Diego, CA (US)

Correspondence Address: NYDEGGER & ASSOCIATES, 348 OLIVE STREET, SAN DIEGO, CA, 92103, US

(21) Appl. No.: 10/774,665

(22) Filed: Feb. 9, 2004

(51) Int. Cl. A61M 25/00

(52) U.S. Cl. 604/525; 264/154

(57) ABSTRACT

An articulation segment for a catheter includes a tube formed with a first plurality of axially aligned slits that are respectively oriented in planes perpendicular to the axis, with each slit extending azimuthally in an arc partway around the axis. The tube is also formed with a second plurality of similarly formed slits that are axially offset and diametrically opposed relative to the slits of the first plurality to allow for a bending of the catheter in a plurality of different planes.

### FIELD OF THE INVENTION

The present invention pertains generally to interventional catheters that are to be advanced into the vasculature of a patient, and to methods for manufacturing such catheters. More particularly, the present invention pertains to catheters that include controllable elements for bending the catheter during the advancement and placement of the catheter in the vasculature. The present invention is particularly, but not exclusively, useful as an articulation segment for a catheter that allows the catheter to bend in a plurality of different planes.

### BACKGROUND OF THE INVENTION

During the advancement of a catheter into the vasculature of a patient, there are several factors that must be taken into consideration. One of the more important considerations is the ability of the catheter to be accurately and properly guided through the vasculature into its intended location or position. An important adjunct of this is the ability of the catheter to be properly configured, if necessary, once it has been properly positioned. In some instances, such as when an over-the-wire catheter is being used, the guideability of the catheter is dependent on the proper pre-positioning of the guidewire in the vasculature. This is not so with other types of catheters. For instance, due to its unique functional refrigeration requirements, a cryocatheter must typically be positioned in the vasculature without the assistance of a guidewire. Furthermore, many catheters, such as cryocatheters, may need to be reconfigured once they have been positioned in the vasculature.

The need for being able to guide a catheter through the vasculature, without the assistance of a guidewire, has been recognized. Heretofore, however, systems for accomplishing this have relied on the catheter's ability to bend in a predetermined plane, and on its ability to be rotated so that the predetermined bending plane can be properly oriented. For example, U.S. Pat. No. 2,574,840 for an invention entitled “Flexible Medical Probe” which issued to Pieri et al., as well as U.S. Pat. No. 5,114,414 which issued to Buchbinder for an invention entitled “Low Profile Steerable Catheter,” both disclose systems for concertedly deflecting the tip, and rotating the body, of a catheter/probe to steer the catheter/probe through the vasculature of a patient.

It happens that, in addition to the ability to guide a catheter through the vasculature, more control over the catheter may be required. New procedures are now being perfected wherein it is necessary for the catheter to be reconfigured after it has been properly positioned in the vasculature. For example, in order to treat atrial fibrillation by cryoablating tissue, it is desirable to configure the tip of the catheter as a ring that can be placed in contact with tissue at an ostium where a pulmonary vein connects with the left atrium. Then, after the tissue around the ostium has been cryoablated, the catheter must again be reconfigured for withdrawal from the vasculature. In this procedure, as in others not mentioned here, there is a need for a catheter that has extensive flexibility for changing configurations.

In light of the above, it is an object of the present invention to provide an articulating segment for a catheter that allows the catheter to be selectively bent in any of several planes without rotating the catheter. Another object of the present invention is to provide an articulating segment for a catheter that allows the catheter to be simultaneously bent in different planes to effectively reconfigure the catheter, as desired. Still another object of the present invention is to provide an articulating segment for a catheter that can bend with a relatively small radius of curvature. Yet another object of the present invention is to provide an articulating segment for a catheter, and a method for its manufacture, that is simple to implement, easy to use, and comparatively cost effective.

### SUMMARY OF THE INVENTION

In accordance with the present invention, an articulation segment for a catheter includes an elongated hollow tube that has a wall and that defines a longitudinal axis. For the present invention, the tube is formed with a first plurality of slits that are cut through the wall and oriented in respective planes that are substantially perpendicular to the axis. Further, each slit extends azimuthally in an arc partway around the axis and each has a center and a substantially same arc length. The respective centers of these slits are aligned with each other in a centerline that is substantially parallel to the axis. Preferably, the tube is a stainless steel hypotube, and the cuts are made through the wall of the tube with widths in a range of approximately ten to five hundred microns. For the present invention this cutting is preferably done using a laser cutting system.

The tube of the present invention also has a second plurality of slits that are formed in substantially the same manner as the first plurality of slits. For a preferred embodiment of the present invention, however, the centerline of the second plurality of slits is diametrically opposed to the centerline of the first plurality of slits. Further, the slits of the first plurality are axially offset from the slits of the second plurality. Thus, as each slit of both the first and second pluralities has a first end and a second end, their respective ends preferably overlap each other. Specifically, the first end of each slit in the first plurality of slits is juxtaposed and overlaps with the second end of adjacent slits in the second plurality of slits. Likewise, the second end of each slit in the first plurality of slits is juxtaposed and overlaps with the first end of an adjacent slit in the second plurality of slits.

In the preferred embodiment of the present invention all of the slits have a substantially same arc length. Generally, this arc length will be greater than one hundred and eighty degrees. Accordingly, the respective ends of the slits in the first and second pluralities of slits will overlap. Preferably, this overlap will be through an arc distance of approximately ten degrees.

In an alternate embodiment of the present invention the first plurality of slits comprise a first set of slits and the second plurality of slits comprise a second set of slits. For this alternate embodiment the tube is further formed with a third set of slits that are coplanar with, and diametrically opposed to, the first set of slits. Further the tube is formed with a fourth set of slits that are coplanar with, and diametrically opposed to, the second set of slits. In this embodiment, the slits in all four sets have a substantially same arc length that is greater than ninety degrees, but less than one hundred and eighty degrees.

As intended for the present invention, within each plurality or set of slits, all of the slits are aligned along a common centerline and they all have a common azimuthal arc length and orientation. For the embodiment of the present invention having only two pluralities or sets of slits, the slits of one plurality are axially offset from the slits of the other plurality and their respective centerlines are azimuthally offset from each other. For the alternate embodiment having four different pluralities or sets of slits, the corresponding slits of diametrically opposed sets are coplanar to each other and are axially offset from the other pair of diametrically opposed sets. In the alternate embodiment, however, the centerlines of adjacent sets are azimuthally offset from each other by an angle of ninety degrees. For either embodiment, the result is a catheter having an articulation segment that is capable of selectively bending the catheter in a plurality of planes.

### BRIEF DESCRIPTION OF THE DRAWINGS

The novel features of this invention, as well as the invention itself, both as to its structure and its operation, will be best understood from the accompanying drawings, taken in conjunction with the accompanying description, in which similar reference characters refer to similar parts, and in which:

FIG. 1 is a perspective view of an articulation segment in accordance with the present invention;

FIG. 2 is a perspective view of a portion of the articulation segment shown in FIG. 1 with portions shown in phantom;

FIG. 3 is a schematic illustration of relative arc lengths and distances pertinent to the articulation segment as shown in FIG. 1 and FIG. 2;

FIG. 4 is a perspective view of a portion of an alternate embodiment of the articulation segment with portions shown in phantom;

FIG. 5A is a perspective view of an articulation segment of the present invention being bent in an x-z plane and an x-y plane; and

FIG. 5B is a perspective view of an articulation segment of the present invention being bent in an y-z plane and an x-y plane.

### DESCRIPTION OF THE PREFERRED EMBODIMENTS

Referring initially to FIG. 1, an articulation segment in accordance with the present invention is shown and generally designated 10. As shown, the articulation segment 10 includes an elongated hollow tube 12 that is formed by a wall 14. In detail, the wall 14 of articulation segment 10 has an outer surface 16, and it has an inner surface 18 that surrounds a lumen 20. As indicated in FIG. 1, when in a straightened configuration, the tube 12 defines a longitudinal axis 22. Preferably, the tube 12 is made of a thermally conductive, rigid material, such as stainless steel, that permits the tube 12 to be rotated around the axis 22.

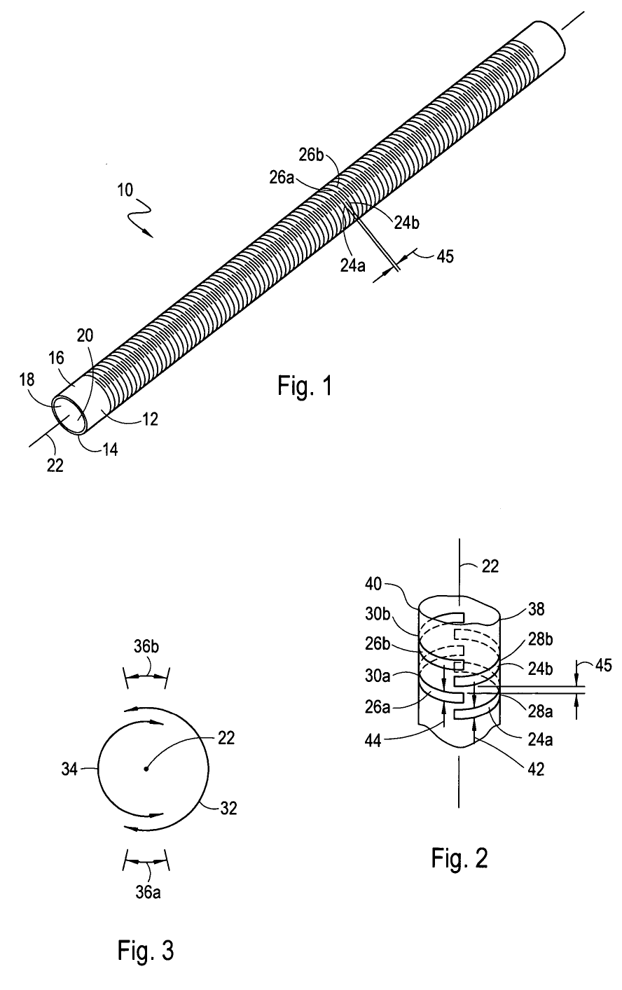

By cross-referencing FIG. 1 with FIG. 2, it will be appreciated that the tube 12 of articulation segment 10 is formed with a first plurality of slits 24, of which the slits 24a and 24b are exemplary. It also has a second plurality of slits 26, of which the slits 26a and 26b are exemplary. Further, the slits 24 have centers 28 (e.g. centers 28a and 28b) and the slits 26 have centers 30 (e.g. centers 30a and 30b) that are respectively midway between the ends of the slits 24, 26. As best appreciated by referencing FIG. 2 with FIG. 3, all of the slits 24 have a substantially same arc length 32 (measured in degrees) and all of the slits 26 have a substantially same arc length 34 (also measured in degrees). Importantly, for the embodiment of the articulation segment 10 shown in FIGS. 1 and 2, the arc lengths 32 and 34 are each approximately greater than one hundred and eighty degrees. Thus, as schematically indicated in FIG. 3, the ends of the slits 24 and 26 will overlap each other through an arc distance 36a or 36b. Preferably, the arc distances 36a and 36b will each be about ten degrees.

In both FIGS. 1 and 2, the slits 24 and 26 are shown to lie in respective planes that are substantially perpendicular to the axis 22. Also, the centers 28 of slits 24 are azimuthally oriented and aligned with each other along a centerline 38, while the centers 30 of slits 26 are similarly oriented and aligned with each other along a centerline 40. As shown in FIG. 2, the centerline 38 is diametrically opposed to the centerline 40. Thus, due to the opposition of their respective centerlines 38 and 40, the slits 24 are azimuthally offset from the slits 26. Also, as evidenced by the overlapping of their respective ends, the slits 24 and slits 26 are axially offset from each other.

As envisioned for the present invention, the plurality of slits 24 (i.e. a set) and the plurality of slits 26 (i.e. a set) will all be cut into the tube 12 by a laser system (not shown). For the embodiment of the articulation segment 10 shown in FIGS. 1 and 2, the slits 24 and 26 extend azimuthally partway around the axis 22 and, preferably, they will have respective widths 42 and 44 that are in a range of from approximately ten to five hundred microns. Also, the axial offset distance 45 between adjacent slits of different sets (e.g. the axial distance 45 between slit 24a and slit 26a in FIG. 1, or FIG. 2) will be in a range of from approximately 200 microns to about 5 millimeters. It will be appreciated, however, that the widths 42, 44 and the axial distances 45 can be varied as required and may fall outside the above-stated ranges.

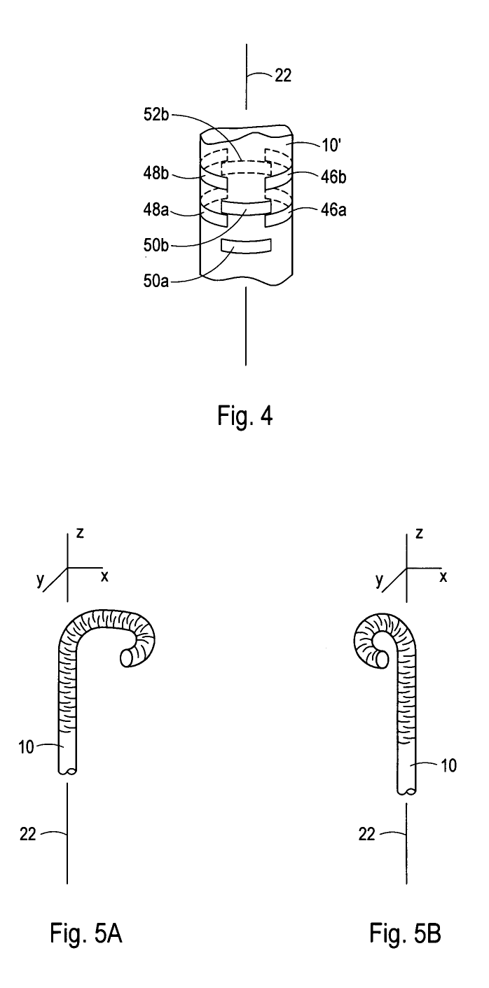

In alternate embodiments of the present invention, there can be three, four or, perhaps even more different sets of slits that are appropriately offset axially and azimuthally from each other. For example, in FIG. 4, the alternate embodiment of an articulation segment 10′ is shown having four different sets of slits. Specifically, a first set (represented by slits 46a and 46b) are shown diametrically opposed, but coplanar, with a second set (represented by slits 48a and 48b). Similarly, a third set (represented by slits 50a and 50b) are shown diametrically opposed, and coplanar, with a fourth set (represented by the slit 52b). Since each set of slits (e.g. slits 46) is coplanar with another set of slits (e.g. slits 48), the arc lengths of the slits in articulation segment 10′ must necessarily be less than one hundred and eighty degrees. Preferably, in order to achieve some overlap (e.g. slit 50a overlaps with both slit 46a and slit 48a) the various slits for the alternate embodiment articulation segment 10′ will have respective arc lengths in a range that is greater than ninety degrees, but less than one hundred and eighty degrees.

For the operation of the present invention, the arrangements of the slits disclosed above allows the articulation segment 10 to be bent simultaneously in different planes. For instance, FIG. 5A shows the articulation segment 10 being bent both in the x-y plane and in the x-z plane. On the other hand, FIG. 5B shows the same articulation segment 10 being bent both in the x-y plane and in the y-z plane. As intended for the present invention, other planar orientations are also possible. The controls for establishing these various orientations for the articulation segment 10 will be dependent on the desires and needs of the operator.

While the particular Catheter Articulation Segment With Alternating Cuts as herein shown and disclosed in detail is fully capable of obtaining the objects and providing the advantages herein before stated, it is to be understood that it is merely illustrative of the presently preferred embodiments of the invention and that no limitations are intended to the details of construction or design herein shown other than as described in the appended claims.

1. An articulation segment of a catheter for selectively bending the catheter in a plurality of planes, the articulation segment comprising: a hollow tube having a wall and defining a longitudinal axis, the tube being formed with a first slit through the wall lying in a plane substantially perpendicular to the axis and extending azimuthally in an arc partway around the axis; the tube being further formed with a second slit through the wall lying in a plane substantially perpendicular to the axis and extending azimuthally in an arc partway around the axis; wherein the first slit is axially and azimuthally offset from the second slit to allow for the selective bending of the catheter.

2. An articulation segment as recited in claim 1 wherein the tube is formed with a plurality of the first slits, with the plurality of first slits being aligned along the axis in a same azimuthal orientation relative to each other; and wherein the tube is formed with a plurality of the second slits, with the plurality of second slits being aligned along the axis in a same azimuthal orientation relative to each other.

3. An articulation segment as recited in claim 2 wherein each first slit has a first end and a second end; wherein each second slit has a first end and a second end; and wherein the first end of the first slit is juxtaposed and overlaps with the second end of an adjacent second slit, and the second end of the first slit is juxtaposed and overlaps with the first end of an adjacent second slit.

4. An articulation segment as recited in claim 3 with each first slit and each second slit having a substantially same arc length, wherein the arc length is greater than one hundred and eighty degrees.

5. An articulation segment as recited in claim 4 wherein the respective ends of the first slits and the second slits overlap through an arc distance of approximately ten degrees.

6. An articulation segment as recited in claim 2 wherein the plurality of first slits comprise a first set of slits and the plurality of second slits comprise a second set of slits; and wherein the tube is further formed with a third set of slits diametrically opposed to the first set of slits with respective slits of the first set lying in a same plane with a corresponding slit of the third set, and further wherein the tube is formed with a fourth set of slits diametrically opposed to the second set of slits with respective slits of the second set lying in a same plane with a corresponding slit of the fourth set.

7. An articulation segment as recited in claim 6 wherein each slit of the first set, second set, third set and fourth set have a substantially same arc length greater than ninety degrees and less than one hundred and eighty degrees.

8. An articulation segment as recited in claim 2 wherein each slit has a width in a range of approximately ten to five hundred microns.

9. An articulation segment as recited in claim 1 wherein the tube is a hypotube made of stainless steel.

10. An articulation segment of a catheter for selectively bending the catheter in a plurality of planes, the articulation segment comprising: a hollow tube having a wall and defining an axis and formed with a plurality of first slits each having a center and a substantially same arc length, with the plurality of first slits cut into the tube through the wall in planes substantially perpendicular to the axis with their respective centers aligned substantially parallel to the axis; the tube being further formed with a plurality of second slits each having a center and a substantially same arc length, with the plurality of second slits cut into the tube through the wall in planes substantially perpendicular to the axis with their respective centers aligned substantially parallel to the axis; wherein the centers of the plurality of first slits are azimuthally offset from the centers of the plurality of second slits, and the plurality of first slits are axially offset from adjacent slits in the plurality of second slits to allow for the selective bending of the catheter.

11. An articulation segment as recited in claim 10 wherein each first slit has a first end and a second end; wherein each second slit has a first end and a second end; and wherein the first end of the first slit is juxtaposed and overlaps with the second end of an adjacent second slit, and the second end of the first slit is juxtaposed and overlaps with the first end of an adjacent second slit.

12. An articulation segment as recited in claim 11 with each first slit and each second slit having a substantially same arc length, wherein the arc length is greater than one hundred and eighty degrees.

13. An articulation segment as recited in claim 12 wherein the respective ends of the first slits and the second slits overlap through an arc distance of approximately ten degrees.

14. An articulation segment as recited in claim 10 wherein each slit has a width in a range of approximately ten to five hundred microns.

15. An articulation segment as recited in claim 10 wherein the tube is a hypotube made of stainless steel.

16. A method for manufacturing an articulation segment for a catheter to allow for a selective bending of the catheter in a plurality of planes, the method comprising the steps of:
providing a hollow tube having a wall and defining a longitudinal axis;
orienting a laser system to cut a plurality of first slits into the tube through the wall with the first slits lying in respective planes substantially perpendicular to the axis and with each first slit having a same azimuthal orientation and extending in an arc partway around the axis and having a center aligned with the centers of other first slits along a first line substantially parallel to the axis; and
using the system to cut a plurality of second slits into the tube through the wall with the second slits lying in respective planes substantially perpendicular to the axis and with each second slit having a same azimuthal orientation and extending in an arc partway around the axis and having a center aligned with the centers of other second slits along a second line substantially parallel to the axis, wherein slits in the plurality of first slits are axially offset from slits in the plurality of second slits, and wherein the fist line is azimuthally offset from the second slit line to allow for the selective bending of the catheter.

17. A method as recited in claim 16 wherein each first slit has a first end and a second end; wherein each second slit has a first end and a second end; and wherein the first end of the first slit is juxtaposed and overlaps with the second end of an adjacent second slit, and the second end of the first slit is juxtaposed and overlaps with the first end of an adjacent second slit.

18. A method as recited in claim 17 with each first slit and each second slit having a substantially same arc length, wherein the arc length is greater than one hundred and eighty degrees and wherein the respective ends of the first slits and the second slits overlap through an arc distance of approximately ten degrees.

19. A method as recited in claim 16 further comprising the steps of:
orienting the system to cut a plurality of third slits into the tube with the third slits being coplanar with the first slits, and with each third slit extending azimuthally in an arc partway around the axis and having a center aligned with the centers of other third slits along a third line substantially parallel to the axis; and
using the system to cut a plurality of fourth slits into the tube with the fourth slits being coplanar with the second slits, and with each fourth slit extending azimuthally in an arc partway around the axis and having a center aligned with the centers of other fourth slits along a fourth line substantially parallel to the axis, wherein the third line is diametrically opposed to the first line and the fourth line is diametrically opposed to the second line to allow for the selective bending of the catheter.

20. A method as recited in claim 19 wherein each slit has a substantially same arc length greater than ninety degrees and less than one hundred and eighty degrees and has a width in a range of approximately ten to five hundred microns.
